(19) United States
(12) Patent Application Publication
(10) Pub. No.: US 2010/0323000 A1
(43) Pub. Date: Dec. 23, 2010

(54) METHOD AND PROCESS FOR PREPARING CARDIOLIPIN

(75) Inventors: Shoukath M. Ali, Vernon Hills, IL (US); Moghis U. Ahmad, Wadsworth, IL (US); Imran Ahmad, Wadsworth, IL (US)

Correspondence Address: WOOD, PHILLIPS, KATZ, CLARK & MORTIMER, 500 W. MADISON STREET, SUITE 3800, CHICAGO, IL, 60661, US

(73) Assignee: NEOPHARM, INC., Lake Bluff, IL (US)

(21) Appl. No.: 12/280,102

(22) PCT Filed: Feb. 22, 2007
(86) PCT No.: PCT/US07/05038

§ 371 Date: Aug. 30, 2010

Related U.S. Application Data
(60) Provisional application No. 60/776,324, filed on Feb. 24, 2006.

(51) Int. Cl. A61K 9/127; C07F 9/10; A61P 43/00; C12N 5/071; A61K 31/6615

(52) U.S. Cl. 424/450; 554/82; 435/325; 514/103

(57) ABSTRACT

The invention provides novel methods for preparing cardiolipin and cardiolipin analogs having varying fatty acid chain lengths, particularly 1,1′,2,2′-tetramyristoyl cardiolipin. The methods comprise reacting a starting compound, such as a 1,2-O-sn-diacylglycerol and a 2-protected glycerol, with a phosphoramidite reagent to produce a protected cardiolipin, which is deprotected to prepare cardiolipin. The cardiolipin and cardiolipin analogs may be prepared in the presence of an activator, such as pyridinium trifluororacetate. The methods of the present invention are used to prepare cardiolipin and cardiolipin analogs in large quantities. The cardiolipin prepared by the present methods can be incorporated into liposomes which can also include active agents such as hydrophobic or hydrophilic drugs. Such liposomes can be used to treat diseases or in diagnostic and/or analytical assays.

### FIELD OF THE INVENTION

The present invention relates to novel methods for the preparation of cardiolipin and cardiolipin analogs. More particularly, the invention relates to methods of preparing cardiolipin and cardiolipin analogs via phosphoramidite chemistry, using an activator. Further, the methods of the present invention are used to prepare cardiolipin and cardiolipin analogs in large quantities.

### BACKGROUND OF THE INVENTION

Cardiolipin (also known as diphosphatidyl glycerol) constitutes a class of complex anionic phospholipids that is typically purified from cell membranes of tissues associated with high metabolic activity, including the mitochondria of heart and skeletal muscles. The negative surface charge of cardiolipin stabilizes liposomes against aggregation-dependent uptake. However, the potential effects of the length and nature (i.e., saturated or unsaturated) of cardiolipin fatty acid chains on liposome aggregation have not been elucidated.

Known methodologies for synthesizing cardiolipin are mainly divided in two groups: (a) coupling the primary hydroxyl groups of a 2-protected glycerol with 1,2-diacyl-sn-glycerol using a phosphorylating agent and (b) condensation at both primary hydroxyl groups of a 2-protected glycerol with phosphatidic acid in the presence of 2,4,6-triisopropylbenzenesulfonylchloride (TPS) or pyridine (See; e.g., Ramirez et al., Synthesis, 11, 769-770 (1976), Duralski at al., Tetrahedron Lett., 39, 1607-1610 (1998), Saunders and Schwarz, J. Am. Chem. Soc. 88, 3844-3847 (1966), Mishina at al., Bioorg. Khim., 11, 992-994 (1985), and Stepanov at al, Zh Org., Khim., 20, 985-988 (1984)). Cardiolipin has also been generated via a reaction between the silver salt of diacylglycerophosphoric acid benzyl ester with 1,3-diiodopropanol benzyl ether or 1,3-diiodopropanol t-butyl ether (See, e.g., De Haas et al., Biochim. Biophys. Acta, 116, 114-124 (1966) and Inoue at al., Chem. Pharm. Bull., 11, 1150-1156 (1963)). Although these methods are suitable for the preparation of analytical quantities of cardiolipin in order to confirm its structure, they are not practical for the routine preparation of large quantities for manufacturing purposes due to the many steps involved, the requirement of careful purification of intermediates and the use of highly photosensitive silver salt intermediates and unstable iodo intermediates.

Phosphate triesters and phosphoramidite esters have been used extensively in nucleic acid synthesis to form phosphate linkages and, to a lesser extent, in phospholipid synthesis (See, e.g., Brownie et al., J. Chem. Soc. Perkin Trans, 1, 653-657 (2000) ). In this respect, Browne et al., supra, describes the preparation of phospholipid analogs, particularly phosphorylcholine analogs, using phosphoramidite methodologies. The phosphatidylinositols PtdIns(4,5)P₂ and PtdIns(3,4,5)P₃, and derivatives thereof, have been prepared using a variety of phosphoramidite reagents, including N,N-diisopropylphosphoramidite (See, e.g., Watanabe et al., Tetrahedron Lett., 35, 123-124 (1994)), difluorenyl phosphoramidite (See, e.g., Watanbe et al., Tetrahedron Lett., 38, 7407-7410 (1997)), and a reagent produced by reacting a diacylglycerol with (benzyloxy)(N,N-diisopropylamino)chlorophosphine (See, e.g., Chen et al., J. Org. Chem., 61, 6305-6312 (1996) and Prestwich et al., Org. Chem. Res., 29, 503-513 (1996)). In addition, phosphotriester analogs of PtdIns(4,5)P₂ and PtdIns(3,4,5)P₃ have been prepared utilizing the phosphoramidite, reagent 2-cyano-ethyl N,N,N,N-tetraisopropylphosphorodiamidite (See, e.g., Gu et al., J. Org. Chem., 61, 8642-8647 (1996)). Moreover, Murakami et al., J. Org. Chem., 64, 648-651 (1999) describes the synthesis of phosphatidyl glycerol from 2,5-dibenzyl-D-mannitol utilizing methyl tetraisopropylphosphorodiamidite as a phosphorylating agent.

Recently, the use of phosphoramidite esters in preparing phospholipids such as cardiolipin, particularly cardiolipin species having varying fatty acid chain lengths, has been reported (See, e.g., Lin et al., Lipids, 39, 285-290 (2004), Krishna et. al., Tetrahedron Lett. 45, 2077-2079 (2004), Krishna et. al., Lipids, 39, 595-600 (2004), Ahmad et. al., U.S. Patent Appl. No. 2005/0181037 A1 and Ahmad et. al., U.S. Patent Appl. No. 2005/0266068 A1). In all of the afore-mentioned synthetic schemes, the first step involves the reaction of 1,2-O-diacyl-sn-glycerol with one or more phosphoramidite reagent(s) followed by coupling with a 2-protected glycerol, wherein a protected cardiolipin is produced. The phosphoramidite reagent(s) in these reactions were activated by 1H-tetrazole.

1H-tetrazole is the most common activator used in the phosphitylation reactions. However, the usage of 1H-tetrazole in large-scale synthesis is limited due to its explosive and highly toxic nature. It requires special handling during its use, disposal and storage. Further, 1H-tetrazole is also very expensive and, therefore, not practical for the cost effective synthesis of cardiolipin.

A need exists for new synthetic methods that can be used to prepare large quantities of saturated and unsaturated cardiolipin species having varying fatty acid chain lengths. A need also exists for new synthetic methods that would increase the availability of a wider variety of cardiolipin species and that would diversify the lipids available for development of new liposomal formulations containing active agents that would include more defined compositions than those currently available.

The present invention provides such methods. These and other advantages of the invention, as well as additional inventive features, will be apparent from the description of the invention provided herein.

### BRIEF SUMMARY OF THE INVENTION

The present invention provides a method for preparing cardiolipin having varying fatty acid chain lengths. The method comprises the steps of: (a) reacting an optically pure 1,2-disubstituted-sn-glycerol with one or more phosphoramidite reagent(s); (b) coupling the product of (a) with a 2-0 protected or 2-0 substituted glycerol in the presence of an activator. The present inventive method can be used to prepare cardiolipin and cardiolipin analogs in large quantities.

### BRIEF DESCRIPTION OF THE DRAWING

FIG. 1 depicts a general scheme for synthesizing cardiolipin;

FIG. 2 depicts a general alternative scheme for synthesizing cardiolipin;

FIG. 3 depicts a general alternative scheme for synthesizing cardiolipin; and

FIG. 4 depicts a scheme for synthesizing 1,1′,2,2′-tetramyristoyl cardiolipin.

### DETAILED DESCRIPTION OF THE INVENTION

The present invention describes methods for preparing cardiolipin variants and analogs having the general formulas I, II and III.

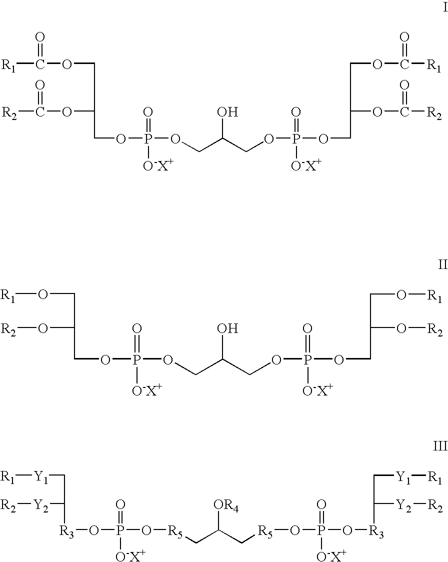

In Formula III, Y₁ and Y₂ are the same or different and are —O—C(O)—, —O—, —S—, —NH—C(O)— or the like. In Formulas I, II and III, R₁ and R₂ are the same or different and are H, saturated and/or unsaturated alkyl group, preferably a C₂ to C₃₄ saturated and/or unsaturated alkyl group. In Formula III, R₃ is (CH₂)ₙ and n=0-15. In Formula III, R₄ is hydrogen, alkyl, substituted alkyl, cycloalkyl, substituted cycloalkyl, a peptide, dipeptide, polypeptide, protein, carbohydrate (such as glucose, mannose, galactose, polysaccharide and the like), heterocyclic, nucleoside, polynucleotide and the like. In Formula III, R₅ is a linker which may (or may not be) added in the molecule depending on the need and applications. However, where added, R₅ can comprise alkyl, substituted cycloalkyl, substituted cycloalkyl, alkoxy, polyalkyloxy (such as pegylated ether containing from about 1 to 500 alkyloxymers ((and can have at least about 10 alkyloxy mers, such as at least about 50 alkyloxy mers or at least about 100 alkyloxy mers, such as at least about 200 alkyloxy mers or at least about 300 alkyloxy mers or at least about 400

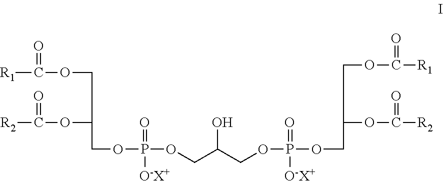

alkyloxy mers)), substituted polyalkyloxy and the like), a peptide, dipeptide, polypeptide, protein, carbohydrate such as glucose, mannose, galactose, polysaccharides and the like. In Formulas I, II and III, X is a non-toxic cation, preferably hydrogen, ammonium, sodium, potassium, calcium, barium ion and the like.

In accordance with the most preferred embodiment, Y₁ and Y₂ in Formula III are —O—C(O)— or —O—, R₁ and R₂ are the same and are a C₂ to C₂₄ saturated and/or unsaturated alkyl group, more preferably between 4 and 18 carbon atoms (such as between about 6 and 14 carbon atoms). R₃ most preferably is CH₂. X most preferably is hydrogen or ammonium ion. In the absence of linker (R₅), the general structure of cardiolipin is disclosed.

The invention provides a method for preparing cardiolipin or an analog thereof of Formulas I, II, or III, comprising reacting an alcohol of the formula IV

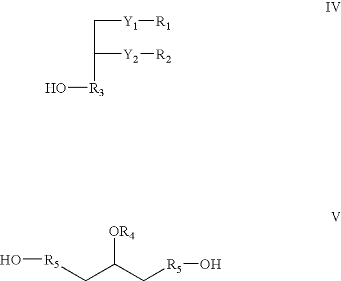

with one or more phosphoramidite reagents and 2-O-protected glycerol or a diol of formula V in the presence of an activator. In Formula IV, R₁, R₂, R₃, Y₁, and Y₂, can be as indicated above with respect to Formulas I, II, or III. In Formula V, R₄ and R₅ can be as indicated above with respect to Formula III. In accordance with the inventive method, the activator can be any suitable pyridinium salt that can facilitate the reaction. Examples of such salts include pyridinium hydrochloride, pyridinium triflate, pyridinium acetate, pyridinium chloroacetate, pyridinium dichloroacetate, pyridinium trichloroacetate and pyridinium trifluoroacetate. In accordance with the inventive method, the coupling phosphoramidites can have a formula of VI or VII:

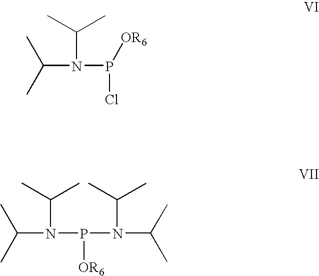

Following the preferred procedure, the invention provides a method for preparing cardiolipin or an analog thereof of formulas I, II, or III. The method comprises the steps of reacting 2-0 protected glycerol or a dial with one or more phosphotriesters in the presence of pyridinium tribromide. Preferred phosphotriesters can be produced by the reaction of an alcohol of formula IV with phosphoramidite of general formula Nail in presence of an activator.

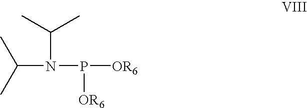

R₆ in Formulas VI, VII, or VIII is a phosphate protecting group, preferably a methyl group, benzyl group or 2-cyanoethyl or silyl group. Other examples of suitable protecting groups include alkyl phosphates including ethyl, cyclohexyl, t-butyl; 2-substituted ethyl phosphates including 2-cyanoethyl, 4-cyano-2-butenyl, 2-(methyldiphenylsily)ethyl, 2-(trimethylsilyi)ethyl, 2-(triphenylsilyl)ethyl; haloethyl phosphates including 2,2,2-trichloroethyl; 2,2,2-tribromoethyl, 2,2,2-trifluoroethyl; benzyl phosphates including 4-chlorobenzyl, fluorenyl-9-methyl, diphertylmethyl and amidates.

In accordance with the inventive method, the preferred activator is pyridinium trifluoroacetate having the formula IX. Pyridinium trifluoroacetate is inexpensive, stable, non-toxic, highly soluble in organic solvents, less acidic and safer to handle than 1-H-tetrazole. However, it is contemplated that any other pyridinium salt can be used including, but not limited to, pyridinium hydrochloride, pyridinium triflate, pyridinium acetate, pyridinium chloro acetate, pyridinium dichloroacetate and pyridinium trichloroacetate.

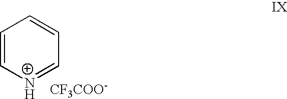

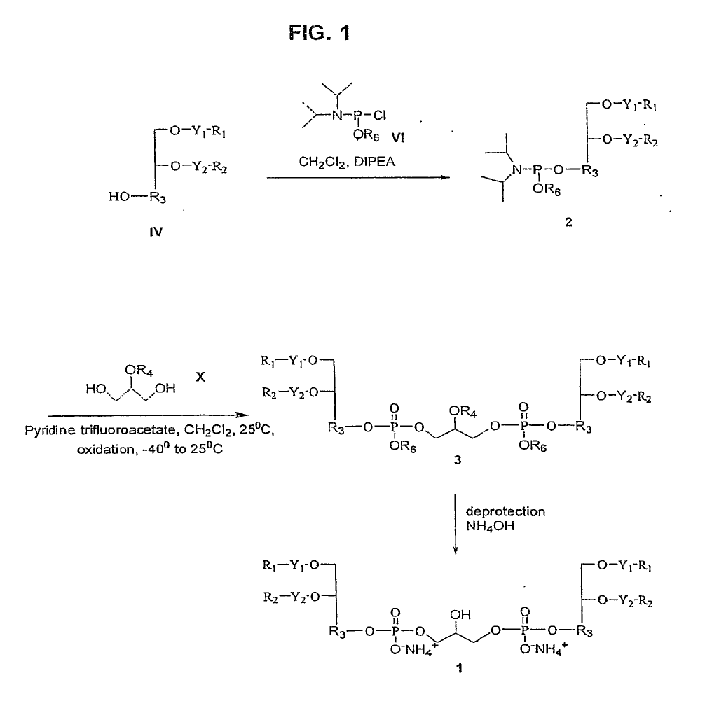

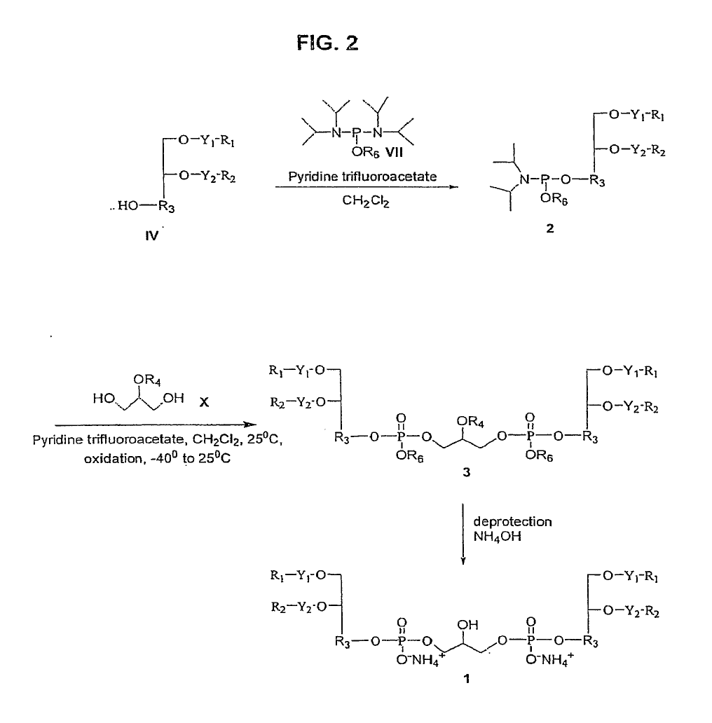

A general sequence of reactions for the synthesis of cardiolipin or an analog thereof in accordance with the present invention is illustrated in FIGS. 1 & 2. The present invention provides a general method for preparing cardiolipin 1 having varying fatty acid chain lengths comprising the steps of: (a) reacting an optically pure 1,2-disubstituted-sn-glycerol IV with one or more phosphoramidite reagent(s) of the general formula VI (FIG. 1) or VII (FIG. 2); (b) coupling the product of (a) 2 with a 2-O-protected glycerol X (R₄ in formula X is a hydroxyl protecting group, preferably an alkyl group or the like, or a silyl protecting group) in a chlorinated solvent (for example, dichloromethane, chloroform or the like) followed by oxidation with m-chloroperoxybenzoic acid (m-CPBA) or hydrogen peroxide or tert-butyl hydroperoxide (t-BuOOH), resulting in the production of a protected cardiolipin 3. Thereafter, deprotection of the protected cardiolipin followed by conversion to an ammonium salt will result in the production of cardiolipin 1 (ammonium salt). The preferred activator in this context of synthetic methods is pyridinium trifluoroacetate.

For reaction with optically pure 1,2-disubstituted-sn-glycerol IV, any suitable phosphoramidite reagent or methodology may be used, such as is described in, for example, Browne et al., supra. Examples of suitable phosphoramidite reagents include N,N-diisopropylmethylphosphonamidic, chloride (See, e.g., Bruzik et al., Tetrahedron Lett., 36:2415-2418 (1995)), (benzyloxy)(N,N-diisopropylamino)chlorophosphine (See, e.g., Prestwich et al. J. Am. Chem. Soc., 113, 1822-1825, (1991)), benzyloxybis (diisopropylamino) phosphine (See, e.g., Dreef et al. Tetrahedron Lett., 29, 6513-6516, (1988); 2-cyanoethyl-N,N,N,N-tetraisopropylphosphoramidite (See, e.g., Browne et al. J. Chem. Soc. Perkin Trans. I. 653-657, (2000)), (2-cyanoethyl)(N,N-diisopropylamino)chlorophosphine (See, e.g., Prestwich et al. J. Org. Chem. 63, 6511-6522, (1998)), difluorenyl diisopropylphosphoramidite (See, e.g., Watanabe et al. Tetrahedron Lett. 38, 7407-7410. (1997)), methyl-N,N,N,N hexane, pentane, heptane, ethyl acetate, chloroform, methylene chloride, methanol and acetone and the like.

Suitable solvents that can be used in the present invention for the crystallization of intermediates and product include hydrocarbons such as pentanes, hexanes, heptanes and the like; ethyl acetate; chlorinated solvents such as methylene chloride, chloroform, 1,2-dichloroethane, and the like; alcohols, for example, methanol, ethanol, isopropanol, n-butyl alcohol, and the like; ketones, for example, acetone, 2-butanone and the like, acetonitrile, tetrahydrofuran toluene, and the like. The solvent for crystallizations can be used as a single solvent or mixture of solvents such as hexane-ethyl acetate, chloroform-acetone, chloroform-methanol, dichloromethane-methanol and the like. When the mixture of solvent is used in the present invention, the ratio of one solvent to another would be 9:1 to 1:9 such as 8:2, 7:3; 6:4; 5:5; 4:6; 3:7; 2:8; 1:9 and the like.

The present invention also provides a convenient process for obtaining intermediates by crystallization with common organic solvents, thereby eliminating the need for extensive column chromatography purification. The final crude product can be purified by column chromatography. One object of the present invention is to provide a process for preparing cardiolipin with at least 80% purity, such as at least 90% pure or at least 95% pure or at least 98% pure or at least 99% or at least 100% pure. Another object of the present invention to provide a process for preparing cardiolipin in a cost effective manner.

The term “alkyl” encompasses saturated or unsaturated straight-chain and branched-chain hydrocarbon moieties. The term “substituted alkyl” comprises alkyl groups further bearing one or more substituents selected from hydroxy, alkoxy (of a lower alkyl group), mercapto (of a lower alkyl group), cycloalkyl, substituted cycloalkyl, halogen, cyano, nitro, amino, amido, imino, thio, —C(O)H, acyl, oxyacyl, carboxyl, and the like.

The inventive method can be used to prepare cardiolipin species comprising fatty acid chains of varying length and saturation. The general structure of a phospholipid fatty acid comprises a hydrocarbon chain and a carboxylic acid group. In general, the length of the fatty acid hydrocarbon chain ranges from about 4 to about 34 carbon atoms; however, the carbon chain is more typically between about 12 and about 24 carbon atoms. In some embodiments, it is desirable for the hydrocarbon chain to comprise, for example, at least about 5 carbon atoms or at least about 10 carbon atoms or even at least about 15 carbon atoms. Typically, the length of the fatty acid hydrocarbon is less than about 30 carbon acids, such as less than about 25 carbon atoms or even less than about 20 carbon atoms. tetraisopropylphosphorodiamidite (See, e.g., Murakami et. al., J. Org. Chem. 64, 648-651 (1999)), dimethyl N,N-diisopropylphosphoramidite (See, e.g., Watanabe et al., Tetrahedron Lett. 34, 497-500 (1993)), dibenzyl diisopropylphosphoramidite (See, e.g., Watanabe et al., Tetrahedron Lett. 41, 8509-8512 (2000)), di-tert-butyl-N,N-diisopropylphosphoramidite (See, e.g., Lindberg et al., J. Org Chem. 67, 194-199, (2002)), 2-(diphenylmethylsily)ethyl-N,N,N,N-tetraisopropylphosphoramidite (See, e.g., Chevallier et al., Org. Lett. 2, 1859-1861, (2000)), (N-trifluoroacetylamino)butyl and (N-trifluoroacetylamino)pentyl-N,N,N,N-tetraisopropylphosphoramidites (See, e.g., Wilk et al., J. Org. Chem. 62, 6712-6713, (1997)).

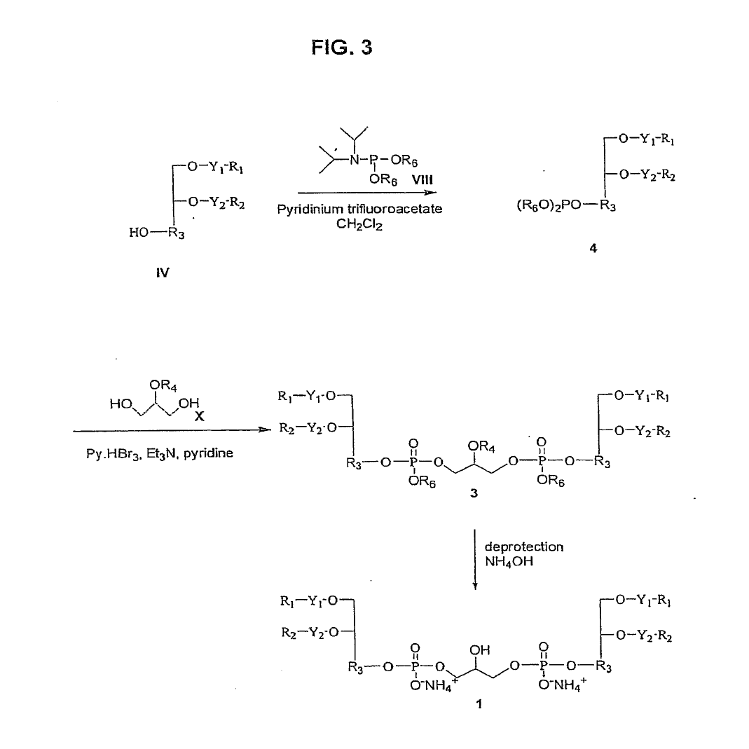

Another embodiment of the present invention is depicted in FIG. 3. In this method, the optically pure 1,2-disubstituted-sn-glycerol IV can be phosphorylated using phosphoramidite VIII and pyridinium trifluoroacetate to yield phosphite triesters 4 which can be coupled with any suitable 2-O-protected glycerol X such as, for example, benzyloxy 1,3-propanediol or 2-levulinoyl-1,3-propanediol using pyridinium perbromide and phosphonium salt methodology (See, e.g., Watanabe et al., supra) to get protected cardiolipin 3. The preferred coupling reagent in this context of synthetic methods is dibenzyl diisopropylphosphoramidite and the preferred activator is pyridinium trifluoroacetate.

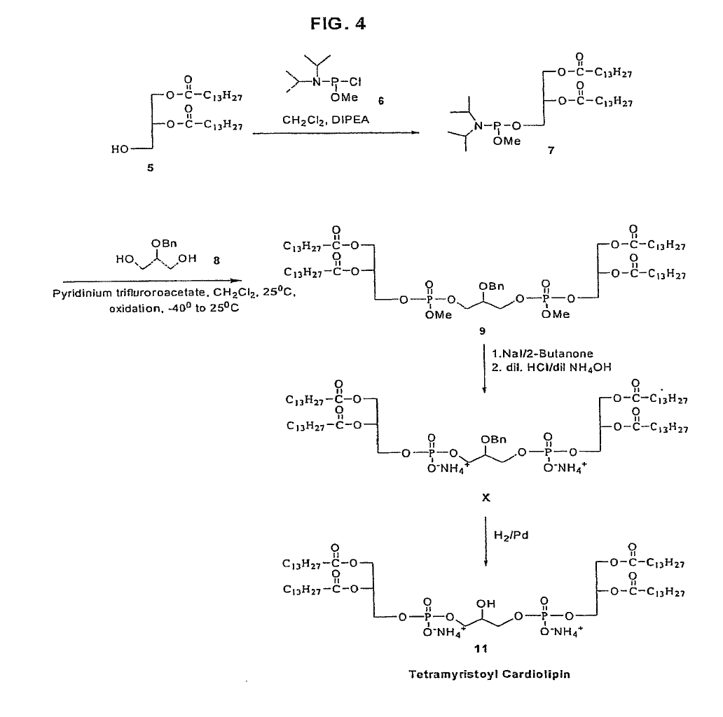

The most preferred method of the present invention is depicted in FIG. 4, wherein the synthetic scheme for 1,1′,2,2′-tetramyristoyl cardiolipin (C_{14:0})) 11 is outlined. The synthesis involves reaction of an optically pure 1,2-dimyristoyl-sn-glycerol 5 with NAT-diisopropylmethylphosphonamidic chloride 6 in the presence of base such as N,N-diisopropylethylamine (DIPEA) in a suitable solvent such as dicholoromethane. The resulting intermediate 7 on coupling in situ with a benzyloxy 1,3-propanediol 8 in the presence of pyridinium trifluoroacetate in a chlorinated solvent (for example dichloromethane, chloroform or the like) followed by oxidation with m-chloroperoxybenzoic acid (m-CPBA) or hydrogen peroxide results in the production of a protected cardiolipin 9. The methyl groups of the protected precursor 9 then is removed by reaction with sodium iodide to produce a sodium salt of cardiolipin, which is then converted to an ammonium salt 10 by treatment with dilute HCl followed by dilute ammonium hydroxide. Deprotection of the benzyl protecting group by catalytic hydrogenation will result in the production of tetramyristoyl cardiolipin 11 (ammonium salt).

The intermediates and final product of the present invention can be purified by column chromatography using a single or a mixture of common organic solvents such as

Most preferably, the cardiolipin prepared by the inventive method comprises a fatty acid chain (i.e., a “short-chain” cardiolipin), and the invention provides a short chain cardiolipin. A short fatty acid chain comprises between about 4 and about 14 carbon atoms and can have between about 6 and about 12 carbon atoms, such as between about 8 and about 10 carbon atoms. Alternatively, the cardiolipin produced by the inventive method can comprise a long chain fatty acid (i.e., a “long-chain” cardiolipin). A long chain fatty acid comprises between about 22 and about 30 carbon atoms, such as between about 24 and about 28 carbon atoms. The inventive method is not limited to the production of short- or long-chain cardiolipin species exclusively. Indeed, it is contemplated that a cardiolipin containing fatty acid chains of intermediate length can also be prepared by the inventive method.

Phospholipid fatty acids typically are classified by the number of double and/or triple bonds in the hydrocarbon chain (i.e., unsaturation). A saturated fatty acid does not contain any double or triple bonds, and each carbon in the chain is bond to the maximum number of hydrogen atoms. The degree of unsaturation of a fatty acid depends on the number of double or triple bonds present in the hydrocarbon chain. In this respect, a monounsaturated fatty acid contains one double bond, whereas a polyunsaturated fatty acid contains two or more double bonds (See, e.g., Oxford Dictionary of Biochemistry and Molecular Biology, rev. ed., A. D. Smith (ed.), Oxford University Press (2000), and Molecular Biology, of the Cell, 3^{rd} ed., B. A. Alberts (ed.), Garland Publishing, New York (1994)). The fatty acid chains of the cardiolipin prepared by the inventive method, whether short or long, also can be saturated or unsaturated.

The described methods can be used to prepare a variety of novel cardiolipin molecules. For example, the methods can be used to prepare cardiolipin variants in pure form containing short or long fatty acid chains. Preferred fatty acids range from carbon chain lengths of about C₂ to C₃₄, preferably between about C₄ and about C₂₄, and include tetranoic acid (C_{4:0}), pentanoic acid (C_{5:0}), hexanoic acid (C_{6:0}), heptanoic acid (C_{7:0}), octanoic acid (C_{8:0}), nonanoic acid (C_{9:0}), decanoic acid (C_{10:0}), undecanoic acid (C_{11:0}), dodecanoic acid (C_{12:0}), tridecanoic acid (C_{13:0}), tetradecanoic (myristic) acid (C_{14:0}), pentadecanoic acid (C_{15:0}), hexadecanoic (palmatic) acid (C_{16:0}), heptadecanoic acid (C_{17:0}), octadecanoic (stearic) acid (C_{18:0}), nonadecanoic acid (C_{19:0}), eicosanoic (arachidic) acid (C_{20:0}), heneicosanoic acid (C_{21:0}), docosanoic (behenic) acid (C_{22:0}), tricosanoic acid (C_{23:0}), tetracosanoic acid (C_{24:0}), 10-undecenoic acid (C_{11:1}), 11-dodecenoic acid (C_{12:1}), 12-tridecenoic acid (C_{13:1}), myristoleic acid (C_{14:1}), 10 pentadecenoic acid (C_{15:1}), palmitoleic acid (C_{16:1}), oleic acid (C_{18:1}), linoleic acid (C_{15:2}), linolenic acid (C_{18:3}), eicosenoic acid (C_{20:1}), eicosdienoic acid (C_{20:2}), eicosatrienoic acid (C_{20:3}), arachidonic acid (cis-5,8,11,14-eicosatetraenoic acid), and cis-5,8,11,14,17-eicosapentaenoic acid, among others. For ether analogs, the alkyl chain will also range from C₂ to C₃₄ preferably between about C₄ and about C₂₄. Other fatty acid chains also can be employed as R₁ and/or R₂ substituents. Examples include saturated fatty acids such as ethanoic (or acetic) acid, propanoic (or propionic) acid, batanoic (or butyric) acid, hexacosanoic (or cerotic) acid, octacosanoic (or montanic) acid, triacontanoic (or melissic) acid, dotriacontanoic (or lacceroic) acid, tetratriacontanoic (or gheddic) aced, pentatriacontanoic (or ceroplastic) acid, and the like; monoethenoic unsaturated fatty acids such as trans-2-butenoic (or crotonic) acid, cis-2-butenoic (or isocrotonoic) acid, 2-hexenoic (or isohydrosorbic) acid, 4-decanoic (or obtusilic) acid, 9-decanoic (or caproleic) acid, 4-dodecenoic (or linderic) acid, 5-dodecenoic (or denticetic) acid, 9-dodecenoic (or lauroleic) acid, 4-tetradecenoic (or tsuzuic) acid, 5-tetradecenoic (or physeteric) acid, 6-octadecenoic (or petroselenic) acid, trans-9-octadecenoic (or elaidic) acid, trans-11-octadecenoic (or vaccinic) acid, 9-eicosenoic (or gadoleic) acid, 11-eicosenoic (or gondoic) acid, 11-docosenoic (or cetoleic) acid, 13-decosenoic (or crude) acid, 15-tetracosenoic (or nervonic) acid, 17-hexacosenoic (or ximenic) acid, 21-triacontenoic (or lumequeic) acid, and the like; dienoic unsaturated fatty acids such as 2,4-pentadienoic (or β-vinylacrylic) acid, 2,4-hexadienoic (or sorbic) acid, 2,4-decadienoic (or stillingic) acid, 2,4-dodecadienoic acid, 9,12-hexadecadienoic acid, cis-9, cis-12-octadecadienoic (or α-linoleic) acid, trans-9, trans-12-octadecadienoic (or liniolelaidic) acid, trans-10,trans-12-octadecadienoic acid, 11,14-eicosadienoic acid, 13,16-docosadienoic acid, 17,20-hexacosadienoic acid and the like; trienoic unsaturated fatty acids such as 6,10,14-hexadecatrienoic (or hiragonic) acid, 7,10,13-hexadecatrienoic acid, cis-6, cis-12-octadecatrienoic (or γ-linoleic) acid, trans-8, trans-10-trans-12-octadecatrienoic (or 3-calendic) acid, cis-8, trans-10-cis-12-octadecatrienoic acid, cis-9, cis-12-cis-15-octadecatrienoic (or α-linolenic) acid, trans-9, trans-12-trans-15-octadecatrienoic (or α-linolenelaidic) acid, cis-9, trans-11-trans-13-octadecatrienoic (or α-eleostearic) acid, trans-9, trans-11-trans-13-octadecatrienoic (or β-eleostearic) acid, cis-9, trans-11-cis-13-octadecatrienoic (or punicic) acid, 5,8,11-eicosatrienoic acid, 8,11,14-eicosatrienoic acid and the like; tetraenoic unsaturated fatty acids such as 4,8,11,14-hexadecatetraenoic acid, 6,9,12,15-hexadecatetraenoic acid, 4,8,12,15-octadecatetraenoic (or moroctic) acid, 6,9,12,15-octadecatetraenoic acid, 9,11,13,15-octadecatetraenoic (or α- or β-parinaric)acid, 9,12,15,18-octadecatetraenoic acid, 4,8,12,16-eicosatetraenoic acid, 6,10,14,18-eicosatetraenoic acid, 4,7,10,13-docasatetraenoic acid, 7,10,13,16-docosatetraenoic acid, 8,12,16,19-docosatetraenoic acid and the like; penta- and hexa-enoic unsaturated fatty acids such as 4,8,19,15,18-eicosapentaenoic (or timnodonic) acid, 4,7,10,13,16-docosapentacnoic acid, 4,8,12,15,19-docosapentaenoic (or clupanodonic) acid, 7,10,13,16,19-docosapentaenoic, 4,7,10,13,16,19-docosahexaenoic acid, 4,8,12,15,18,21-tetracosahexaenoic (or nisinic) acid and the like; branched-chain fatty acids such as 3-methylbutanoic (or isovaleric) acid, 8-methyldodecanoic acid, 10-methylundecanoic (or isolauric) acid, 11-methyldodecanoic (or isoundecylic) acid, 12-methyltridecanoic (or isomyristic) acid, 13-methyltetradecanoic (or isopentadecylic) acid, 14-methylpentadecanoic (or isopalmitic) acid, 15-methylhexadecanoic, 10-methylheptadecanoic acid, 16-methylheptadecanoic (or isostearic) acid, 18-methylnonadecanoic (or isoarachidic) acid, 20-methylheneicosanoic (or isobehenic) acid, 22-methyltricosanoic (or isolignoceric) acid, 24-methylpentacosanoic (or isocerotic) acid, 26-methylheptacosanoic (or isomonatonic) acid, 2,4,6-trimethyloctacosanoic (or mycoceranic or mycoserosic) acid, 2-methyl-cis-2-butenoic(angelic)acid, 2-methyl-trans-2-butenoic (or tiglic) acid, 4-methyl-3-pentenoic (or pyroterebic) acid and the like.

The term ‘hydroxyl protecting group’ used herein refers to the commonly used protecting groups disclosed by T. W. Greene and P. G. Wuts, Protective Groups in Organic Synthesis, 3rd edition, John Wiley & Sons, New York (1999). Such protecting groups include methyl ether, substituted methyl ethers including methoxymethyl, benzyloxymethyl, p-methoxybenzyloxymethyl, 2-methoxyethoxymethyl, tetrahydropyranyl, tetrahydrofuranyl ethers; substituted ethyl ethers like 1-ethoxyethyl, 1-methyl-1-benzyloxyethyl, allyl, propargyl; benzyl and substituted benzyl ethers including p-methoxybenzyl, 3,4-dimethoxybenzyl, triphenylmethyl; silyl ethers including trimethylsilyl, triethylsilyl, t-butyldimethylsilyl, t-butyldiphenylsilyl, diphenylmethylsilyl; esters including formate, acetate, chloroacetate, dichloroacetate, trichloroacetate, benzoate, levulinylate and carbonates.

The term ‘phosphate protecting group’ used herein refers to the commonly used protecting groups described by T. W. Greene and P. G. Wuts, Protective Groups in Organic Synthesis, 3rd edition, John Wiley & Sons, New York (1999). Such protecting groups include alkyl phosphates including methyl, ethyl, cyclohexyl, t-butyl; 2-substituted ethyl phosphates including 2-cyanoethyl, 4-cyano-2-butenyl, 2-(methyldiphenylsily)ethyl, 2-(trimethylsilyl)ethyl, 2-(triphenylsilyl)ethyl; haloethyl phosphates including 2,2,2-trichloroethyl, 2,2,2-tribromoethyl, 2,2,2-trifluoroethyl; benzyl phosphates including 4-chlorobenzyl, fluorenyl-9-methyl, diphenylmethyl and amidates.

The cardiolipin molecules described herein and cardiolipins produced by the inventive method can be used in lipid formulations. Complexes, emulsions and other formulations including the inventive cardiolipin also are within the scope of the present invention. Such formulations according to the present invention can be prepared by any suitable technique. In addition to the inventive cardiolipin, the liposomal composition, complex, emulsion and the like can include stabilizers, absorption enhancers, antioxidants, phospholipids, biodegradable polymers and medicinally active agents among other ingredients. In some embodiments, it is preferable for the inventive composition, especially liposomal composition, to include one or more targeting agents, such as a carbohydrate, protein or other ligand that binds to a specific substrate, such as an antibody (or fragment thereof) or ligand that recognizes cellular receptors. The inclusion of such agents (such as carbohydrate or one or more proteins selected from the group consisting of antibodies, antibody fragments, peptides, peptide hormones, receptor ligands such as an antibody to a cellular receptor and mixtures thereof) can facilitate the targeting of a liposome to a predetermined tissue or cell type.

The following example further illustrates the invention but, of course, should not be construed as in any way as limiting its scope.

### Example 1

This example demonstrates a method for preparing 1,1′,2,2′-tetramyristoyl cardiolipin 11. The compound 11 can be synthesized via the synthetic route outlined in FIG. 4. To a solution of 1,2-O-dimyristoyl-sn-glycerol 5 (148 g, 282.20 mmol) and dry

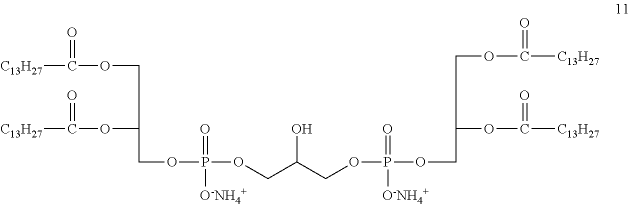

N,N-diisopropylethylamine (59 mL, 338.8 mmol) in CH₂Cl₂ (1.7 L) was added dropwise N,N-diisopropylmethylphosphonamidic chloride 6 (59 g, 299 mmol) at room temperature over 30 minutes. After the reaction mixture was stirred at room temperature for 2 hours, pyridinium trifluoroacetate (65.6 g, 339.2 mmol) was added. To this reaction mixture, a solution of 2-benzyloxy-1,3-propanediol 8 (25 g, 137.20 mmol) in CH₂Cl₂ (280 mL) was added dropwise. The reaction mixture was stirred at room temperature for 3 hours. The reaction mixture was then cooled to 5-10° C. (internal temperature) and a solution of 35 w % hydrogen peroxide (35 mL, 424.8 mmol) was added such that the temperature of the reaction mixture was kept below 10° C. On warming to 25° C., the mixture was transferred to a separating funnel and washed with 10% sodium thiosulfate solution (340 mL), water (2×500 mL), brine (2×500 mL). The organic phase was concentrated in vacuo to yield an oil residue. The crude oil was triturated in acetonitrile (3.5 L) for 30 minutes then stored in freezer (−20° C.) for 24 hours. The solids were filtered over a cold-finger fritted (10-20 μm) glass funnel (cool to −30° C. using dry ice/acetone) under vacuum. The solids were transferred from fritted funnel to a 4 L flask. Heptane (2 L) was added and triturated for 30 minutes before storing in the freezer (−20° C.) for 24 hours. The solids were filtered over a cold-finger fritted (10-20 μm) glass funnel (cool to −30° C. using dry ice/acetone) under vacuum. The solids were collected by dissolving it in hexane. The solvents were removed to provide 174 g of 2-O-Benzyl-1,3-bis(1,2-O-dimyristoyl-sn-glycero-3-phosphoryl)glycerol dimethyl ester 9 as a colorless oil. R_{f}0.27 (hexane-ethyl acetate, 1:1 by vol.).

To a stirred solution of fully protected cardiolipin 9 (174 g) in 2-butanone (1.74 L) was added NaI (48.02 g), and the reaction mixture was refluxed for 1.5 hours and cooled to 25° C. and then at −20° C. for 2 hours. The resulting white precipitate was filtered and washed with cold (−20° C.) acetone (400 mL). The disodium salt was converted to its corresponding ammonium salt by dissolving it in ethyl acetate (1.6 L) and 0.5M HCl (720 mL) and stirred at room temperature for 1 hour. The organic layer was separated and washed with H₂O. The organic layer was neutralized by addition of 5 M NH₄OH (120 mL). The stirring was continued for 15 minutes and then stored in a freezer for 1 hour. The solids were filtered through a fritted (10-20 μm) glass funnel under vacuum and washed with cold (−20° C.) acetone (400 mL). The solids were collected by dissolving it in hexane. The solvents were dried under vacuum to afford 159 g of 2-O-benzyl-1,3-bis(1,2-O-dimyristoyl-sn-glycero-3-phosphoryl)glycerol diammonium salt 10 as a white solid. R_{f} 0.53 (CHCl₃/MeOH/NH₄OH, 65/25/5 by vol.). 2-O-benzyl-1,3-bis(1,2-O-dimyristoyl-sn-glycero-3-phosphoryl)glycerol diammonium salt 10 (159.0 g) was dissolved in ethyl acetate (500 mL) at 30° C. for 1 hour. The solution was filtered through a 0.2 μm PTFE membrane under vacuum. The filtrate was transferred into a 2 L hydrogenation pressure vessel and diluted with ethyl acetate (500 mL). 10% Pd—C (64 g) was added and the mixture was stirred on the hydrogenator at 50 psi for 16 hours. Filter the precipitated product and catalyst over Celite (865 g) and discarded the filtrate. Washed the Celite cake three times with 50% methanol in chloroform (3×6 L) and concentrated the washings and dried under high vacuum. The crude product was purified over silica gel column (2.5 kg) by eluting first with CHCl₃:MeOH:NH₄OH (100:15:1, 4 L) and then with CHCl₃:MeOH:NH₄OH (65:15:1, 23 L). The fractions containing the pure products were pooled and filtered through a 0.2 μm PTFE membrane. The solvents were removed and dried under high vacuum to give 72 g (overall yield 41%) of 1,3-bis(1,2-O-dimyristoyl-sn-glycero-3-phosphoryl)glycerol diammonium salt (tetramyristoyl cardiolipin) 11 was obtained. TLC (CHCl₃/MeOH/NH₄OH 65:25:5) R_{f}=0.29; ¹H NMR (500 MHz, CDCl₃) δ 7.32 (br s, NH₄), 5.26 (m, 2H, RCOOCH), 4.34-3.92 (m, 13H, RCOOCH₂, POCH₂, HOCH), 2.33 (m, 8H, —CH₂COO—), 2.29 (t, J=7.5, 1H, CHOH), 1.58 (m, 8H, —CH₂CH₂COO—), 1.30 (br s, 80H, CH₂), 0.88 (t, J=6.5, 12H, CH₃); FTIR (ATR) 3231 s, 2918 s, 2850 s, 1738 s, 1467 w, 1378 w, 1203 ms, 1067 s cm⁻¹; ESI-MS, m/z (M−2NH₄)²-619.9, (M−2NH₄—RCOO)⁻ 1011.9, (M−2NH₄₎⁺-1-1)⁻ 1240.2.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. Drummond, D. C.; Meyer, O.; Hong, K.; Kirpotin, D. B.; Papahadgopoulos, D. Pharm. Rev., 1999, 51, 691-743.
2. Ramirez, F.; Ioannou, P. V.; Marecek, J. F.; Golding, B. T.; Dodd, G. H. Synthesis. 1976, 11, 769-770.
3. Duralski, A. A.; Spooner, P. J. R.; Watts, A. Tetrahedron Lett. 1989, 30, 3585-3588.
4. Duralski, A. A.; Spooner, P. J. R.; Rankin, S. E.; Watts, A. Tetrahedron Lett. 1998, 39, 1607-1610.
5. Saunders, R. M.; Schwarz. J. Am. Chem. Soc. 1966, 88, 3844-3847.
6. Mishina, I. M.; Vasilenko, A. E.; Stepanov, A. E.; Shvets, V. I. Bioorg. Khim. 1985, 11, 992-994.
7. Stepanov, A. E.; Makarova, I. M.; Shvets, V. I. Zh. Org., Khim. 1984, 20, 985-988.
8. DeHaas, G. H.; Bonsen, P. P. M.; VanDeenen, Biochim. Biophys. Acta, 1966, 116: 114-124.
9. Inoue, K.; Sahara, Y.; Nojima, S. Chem. Pharm. Bull., 1963, 1150-1156.
10. Browne, J. E.; Driver, M. Russel, 7. C.; Sammes, P. G. J. Chem. Soc. Perkin. Trans. I. 2000, 653-657.
11. Watanabe, Y.; Inada, E.; Jinno, M.; Ozaki, S. Tetrahedron Lett. 1993, 34, 497-500.
12. Watanabe, Y.; Hirofuji, H.; Ozaki, S. Tetrahedron Lett. 1994, 35, 123-124.
13. Watanabe, Y.; Nakamura, T.; Mitsumoto, H. Tetrahedron Lett. 1997, 38, 7407-7410.
14. Watanabe, Y.; Ishikawa, H. Tetrahedron Lett. 2000, 41, 8509-8512.
15. Watanabe, Y.; Nakatomi, M. Tetrahedron Lett. 1998, 39, 1583-1586.
16. Chen, J.; Feng, L.; Prestwich, G. D. J. Org. Chem. 1998, 63, 6511-6522.
17. Lindberg, J.; Ekeroth, J.; Konradsson, P. J. Org. Chem. 2002, 67, 194-199.
18. Chen, J.; Profit, A. A.; Prestwich, G. D. J. Org. Chem. 1996, 61, 6305-6312.
19. Prestwich, G. D. Acc. Chem. Res. 1996, 29, 503-513.
20. Gu, Q. M.; Prestwich, G. D. J. Org. Chem. 1996, 61, 8642-8647.
21. Murakami, K.; Molitor, E. J.; Liu, H. W. J. Org. Chem. 1999, 64, 648-651
22. Lin, Z.; Ahmad, M. U.; Ali, S. M.; Ahmad, I. Lipids, 2004, 39, 285-290.
23. Krishna, U. M.; Ahmad, M. U.; Ahmad, I. Tetrahedron Lett. 2004, 45, 2077-2079.
24. Krishna, U. M.; Ahmad, M. U.; Ali, S. M.; Ahmad, I. Lipids, 2004, 39, 595-600.
25. Ahmad, M. U.; Lin, Z.; Ali, S. M.; Ahmad, I. US. Patent No. 20050181.037 A1; WO 03/099830 A2.
26. Ahmad, M. U.; Krishna, U. M.; Ahmad, I. US. Patent No. 20050266068 A1; WO 04/039817 A1.
27. Sanghvi, Y. S.; Guo, Z.; Pfundheller, H. M.; Converso, A. Org. Proc. Res. Dev., 2000, 4, 175-181.
28. Eleuteri, A.; Capaldi, D. C.; Krotz, A. H.; Cole, D. L.; Ravikumaz, V. T. Org. Proc. Res. Dev., 2000, 4, 182-189.
29. Sanghvi, Y. and Manoharan, M. U.S. Pat. No. 6,274,725 B1.
30. Prestwich, G. D.; Marecek, J. F.; Mourey, R. J.; Thiebert, A. B.; Ferris, C. D.; Danoff, S. K.; Snyder, S. H. J. Am. Chem. Soc. 1991, 113, 1822-1825.
31. Dreef, C. E.; Elie, C. J. J.; Hoogerhout, P.; van der Marel, G. A.; van Boom, J. H. Tetrahedron Lett. 1988, 29, 6513-6516.
32. Inoue, K.; Nojima, S. Chem. Pharm. Bull. 1968, 16, 76-81.
33. Ioannou, P. V.; Marecek, J. F. Chem. Chron. 1986, 15, 205-220.
34. Ramirez, F.; Ioannou, P. V.; Marecek, J. F.; Dodd, G. H.; Golding, B. T. Tetrahedron. 1977, 33, 599-608.
35. Mishina, I. M.; Vasilenko, A. F.; Stepanov, A. E.; Shvets, V. I. Bioorg. Khim. 1987, 13, 1110-1115.
36. Keana, J. F. W.; Shimiju, M.; Jernstedt, K. K. J. Org. Chem. 1986, 51, 2297-2299.
37. Chevallier, J.; Sakai, N.; Robert, F.; Kobayashi, T.; Gruenberg, J.; Matile, S. Org. Lett. 2000, 2, 1859-1861.
38. Wilk, A.; Srinivasachar, K.; Beaucage, S. J. Org. Chem 1997, 62, 671.2-6713.
39. Moriguchi, T.; Yanagi, T.; Kunimori, M.; Wada, T.; Sekine, M. Org. Chem. 2000, 65, 8229-8238.

1. A method for preparing a cardiolipin analog of formulas I, II or III 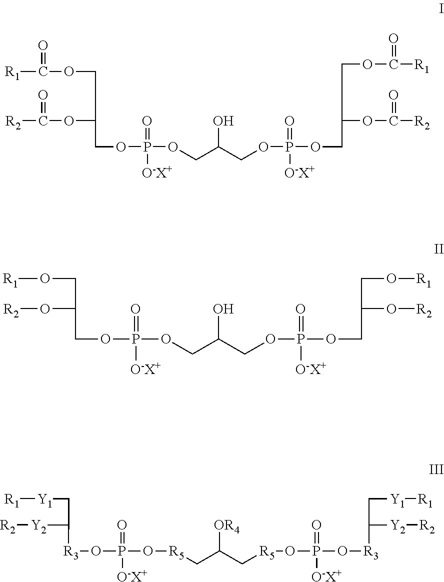
comprising reacting an alcohol of the formula IV 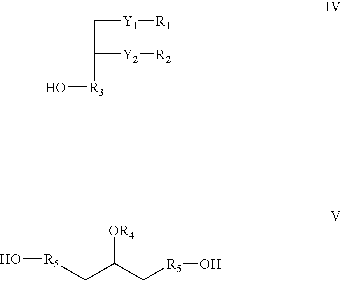
with one or more phosphoramidite reagents and 2-O-protected glycerol or 2-O-substituted glycerol in the presence of an activator, wherein, in Formulas I, II, III, or IV
Y₁ and Y₂ are the same or different and are —O—C(O)—, —O—, —S—, or —NH—C(O)—;
R₁ and R₂ are the same or different and are H, C₂ to C34 saturated or unsaturated alkyl group;
R₃ is (CH2)ₙ and n 0-15;
R₄ is hydrogen, alkyl, substituted alkyl, cycloalkyl, substituted cycloalkyl, a peptide, dipeptide, polypeptide, protein, carbohydrate, heterocyclic, nucleoside, polynucleotide;
R₅ is a linker comprising alkyl, substituted alkyl, cycloalkyl, substituted cycloalkyl, alkyloxy, polyalkyloxy, a peptide, dipeptide, polypeptide, protein, carbohydrate; and
X is a non-toxic cation.

2. The method of claim 1 wherein at least one of the coupling phosphoramidites is of formula VI.

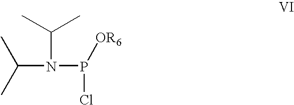

3. The method of claim 1 wherein at least one of the coupling phosphoramidites is of formula VII.

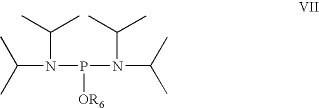

4. A method for preparing cardiolipin or an analog thereof of formulas I, II, or III comprising reacting 2-O protected glycerol with one or more phosphotriesters in the presence of pyridinium tribromide.

5. The method of claim 4, wherein one or more of the phosphotriesters are produced by reacting an alcohol of formula IV with phosphoramidite of general formula VIII in presence of an activator.

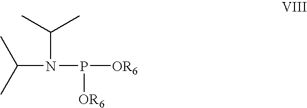

6. The method claim 1 or 5, wherein the activator is selected from the group consisting of pyridinium hydrochloride, pyridinium triflate, pyridinium acetate, pyridinium chloroacetate, pyridinium dichloroacetate, pyridinium trichloroacetate and pyridinium trifluoroacetate.

7. The methods of any of claim 1 or 5, wherein the preferred activator is pyridinium trifluororacetate having formula IX

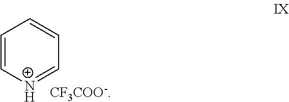

8. The methods of any of claim 2, 4, or 5, wherein R₆ in formulas VI, VII, or VIII is a phosphate protecting group including alkyl phosphates including methyl, benzyl, ethyl, cyclohexyl, t-butyl; 2-substituted ethyl phosphates including 2-cyanoethyl, 4-cyano-2-butenyl, 2-(methyldiphenylsilyl)ethyl, 2-(trimethylsilyl)ethyl, (triphenylsilyl)ethyl; haloethyl phosphates including 2,2,2-trichloroethyl, 2,2,2-tribromoethyl, 2,2,2-trifluoroethyl; benzyl phosphates including 4-chlorobenzyl, fluorenyl-9-methyl, diphenylmethyl and amidates.

9. The methods of any of claim 2, 3 or 5, wherein the preferred phosphate protecting groups are methyl, benzyl group or cyanoethyl group.

10. The method of claim 1, wherein at least one of R₁ and/or R₂ is a saturated or unsaturated alkyl group having between 2 and 34 carbons.

11. The method of claim 10, wherein the cardiolipin comprises short-chain fatty acids having between 4 and 18 carbons.

12. The method of claim 10 or 11, wherein the cardiolipin has between 6 and 14 carbons.

13. The method of claim 1, wherein the cardiolipin comprises long-chain fatty acids having between 14 and 34 carbons.

14. The methods of any of claims 1-13, wherein the cardiolipin is saturated and/or unsaturated.

15. A cardiolipin or cardiolipin analog prepared by the method of claim 1.

16. A method of preparing 1,1′,2,2′-tetramyristoyl cardiolipin of formula 11 comprising reacting 1,2-dimyristoyl-sn-glycerol of formula 5 with a phosphoramidite reagent of formula 6 and 2-O-protected glycerol of the formula 8 in presence of an activator followed by the removal of protecting groups.

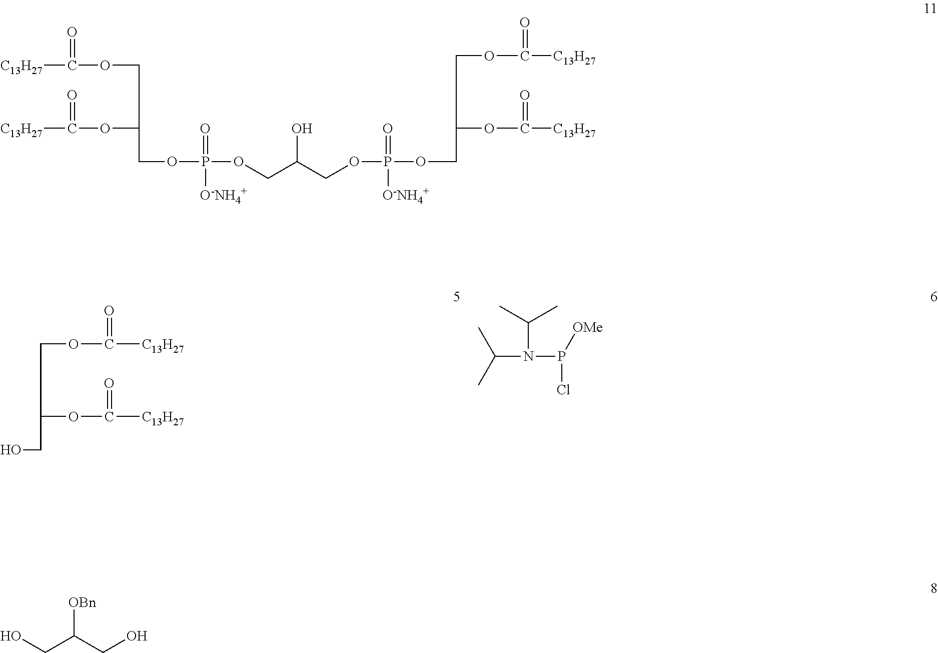

17. The method of claim 16, wherein the activator is pyridinium trifluoroacetate.

18. The methods of any of claim 1, 4 or 16, wherein intermediates and the final products are purified by crystallization and/or column chromatography techniques.

19. The method of claim 18, wherein the crystallization arid/or column chromatography is done using a single or a mixture of common organic solvents.

20. The method of claim 19, wherein the common organic solvents are selected from the group consisting of pentane, hexane, heptane, dichloromethane, chloroform, 1,2-dichloroethane, ethyl acetate, ethanol, methanol, isopropanol, acetone, 2-butanone, tetrahydrofuran, acetonitrile and toluene.

21. A method of preparing a liposome, comprising preparing a cardiolipin or a cardiolipin analog by the method of claim 1 including said cardiolipin or cardiolipin analog in a liposome.

22. A liposome composition comprising a cardiolipin or a cardiolipin analog prepared by the method of claim 1 and an active agent in said composition.

23. A method for treating a human disease or animal disease, comprising preparing cardiolipin or a cardiolipin analog by the method of claim 1 and including said cardiolipin or cardiolipin analog complexed with an active agent and administering said liposome to a subject on need thereof.

24. A method of delivering an active agent to a cell, comprising preparing a cardiolipin or cardiolipin analog by the method of claim 1 including said cardiolipin or cardiolipin analog and an active agent in a liposome and delivering said liposome to a cell.
